SEQUENCE LISTING (13 sequences)

SEQ ID NO: 1 - DNA, 4300 bp, Rhodococcus erythropolis
gggccggaggccctacgctccgtacaccggcgtcggaatctccgccttctccaacagcgttgccacgacgggcccgatctccttcgggtcccaacggtcacccttgtcctcgctcgggccgtgacgccagccctccgcgacggtgatctttccgccctcgacctcgaacacccgccccgtgacgtccttggactcggcgctaccgagccagacgaccagcggcgagatgttctcgggcgccatggcatcgaagccctcttcgggagctgccatcgactcggccatcgccccacccgcgccgacggtcatgcgcgtgcgcgcagcaggcgcgatcgcgttgacactgacgccgtaattcttgagctcggctgcggcctggatggtcatctcggcgataccggccttcgccgcagcgtagttaccctggccgatcgaaccttgcagtcctgcacccgaactggtgttgatgatgcgtgcgtcgacggtcttgcctgccttggcctcagcgcgccagtaggcagcagcgtgacgcagcggcgcgaagtgccccttgaggtgcacgcgaatcaccgcgtcccactcgccttcgctcatgccgaccagcatgcggtcccgcaggaagcccgcgttgttcacgagtacgtccaggccaccgaaggtgtcgatcgcggtcttgatcagattctcggcgcccgcccagtcggcgacgtcgtctccgttgaccactgcttggccaccggcggcgatgatctccgcgacaacctgctcggccggactctcaccggtctcggaaccgtcggcacccgcaccgatgtcgttgacgacgaccttggcgccctcggcagcaaacgccaaggcatgcgcacgtccgatcccgcgaccagccccggtgatgatgactacgcgtccgtcgaccaatccactcatatttgtctcctaatgcactctgctcgggcttgtcggctccgcttgtgtgctaacttaccaagcaatcgcttggtttggtacacagacgtaaggaatcccgatatgggcatcagcacctcctccgacggaaccggcatcaccacggtcaccatcgactacgcgccggtcaatgcaatcccgtccaagggatggttcgagcttgccgacgccatcctcgacgcgggcaaggatctcaacactcacgtggtgatcctgcgagccgagggacgcggattcaacgccggcgtcgacatcaaggagatgcaggcgaccgagggattcgacgcactggtcgccgccaaccgcggttgcgccgcggccttcgcagccgtctacgactgcgctgtacccgtcgtcgtggccgtcaacggattctgtgtcggcggtggcatcggactggtcggcaacgcggacgtgatcgtcgcttccgacgacgcgatcttctccctccccgaggtcgaccgcggcgcactcggcgctgccacacacctctcacggctggtgccgcagcacatgatgcgcacgctctactacaccgcacagagcgtcgacgcccacacactcaaacagttcggcagcgtctacgacgtggtcccgcgcgagaagctcgacgagtgcgctcgcgagatcgccgcaaagatcgccgcgaaggacacccgcatcatccggtgcgccaaggaagccatcaacggcatcgacccggtcgacgtcaagggcagctaccgcctcgagcagggctacaccttcgaactcaacctcctcggtgtgtccgacgaacaccgcgacgaattcgtcgccaccggcaagccgcgcgagaacgcaacccagaaggaaggctgagacaaacatggctagcaagcgtgacaagacgaagtcgctcgacgaggtagtcggcgagctccgcagcggaatgacgatcggcttgggcggctggggatctcgccgcaagccgatggccttcgtgcgtgcgattctgcgttcggacatcaaggacctgaccgtggtcacttacggcggaccggatctcgggctgctgtgctcggccggcaaggtcaagaaggcgtactacggtttcgtgtccctggactccgctccgttctacgatccgtggtttgccaaggcacgcaccgccggtgagatcgaagtccgcgagatggacgagggaatggtcaagtgcggcctcgaagccgctgccgctcgcctccctttcctcccgatccgcgcgggcctgggttcggacgttcgcaacttctggggcgacgaactcaagaccgtcacctccccgtacccagaggccgacggccgttccgagaccttgatcgcgatgcctgcactcaacctcgatgcgtcgttcgttcacctcaatctcggtgacaagcacggcaatgccgcgtacaacggtgtcgacccgtacttcgacgacctgtactgcatggccgccgaaaagcgttacgtctccgtcgaacgcattgtcgagaccgaggaactggtcaaatccgttccactgcagaatcttctgctcaaccgcatgatggtcgacgccgtcgtcgaggccccgaacggtgcccacttcacgttggccggcgaaagctacggccgcgacgagaagttccagcgtcactacgccgaggccgccaagacccccgagtcgtggcagacgttcgtcgacaccttcctctccggcagcgaagaggactaccaagccgcagtaaagaagtttgccgattcatcaaaggcaggggagcaggcaaaatgagcgaatccaccgtcacccgcgcagagtacgttgttctcgcgtgcgctgaaatcttctccggtgcaggcgaaatcatggccagcccgatgtcgacgtcgtccaccatcggcgctcgcctggctcggctcaccaccgaacccgacctgctgatcaccgatggtgaagccctcattctcgaggacaccccggcagtcggaacgaagggccccatcgaaggatggatgcctttccgcaaggtgttcgacgtcgtcgcttcgggccgtcgccacgtagtcatgggcgccaatcagctcgatcgccacggcaaccagaacctctccgccttcggcccgcttcagcagccgacgcgtcagatgttcggtgtgcgcggcgccccgggcaacaccatcaaccacgcgacgagctacttcgtccccaagcactccaagcgagtgttcgtcgacaaggtcgacgtggtgtgcggtgtcggctacgaccagatcgatcccgagaacccggcatacaagtacctgaacatcccccgcgttgtcaccaacctcggtgtcttcgacttcggtggaccgggaaacaccttccgcgcgctgagcctccatcccggcgtcaccgccgaagaggtagccgagaacacctcgttcgaggtagccggactcgccgaggccggtatcacccgtgaccccaccgccgaagagctccacctcattcgcgagaccctcgatccgcgcaaccttcgggaccgtgaggtctcggcatgacccctactctgaagacagcactgaccgaactggtcggtgtcgagtacccgatcgtgcagacgggcatgggctgggtttccgggccagcgctgacgtcggcgacggccaacgcgggcggtctcggcatcctggcttcggccacgatgacctacgacgagctcgagcacgcgatcaagaagacgaagcagctcacggacaagccgttcggtgtcaacatgcgtgccgacgccaccgacgcgccgcagcgcgcggacctgctgatccgtgagggcgtcaaggtcgcgtcgtttgccttggctcccaagaaggaactgatcgccaagctcaaggaccacggcatcgtcgtcgtaccgtcgatcggtgccgcgaagcatgcggtcaaggttgcatcttggggcgcggacgccgtcatcgtccagggcggcgaaggtggtggtcacaccggcggtgtggcaacgacattgcttcttccctcggttctcgacgcggtcgatattccggtcatcgccggtggcggattcttcgacggccgcgggctggccgcagcgttggcgtacggcgctgccggtgtcgccatggggacgcgtttcttgctcaccagtgactcttcggtgccggactccgtcaagcaggaatacctcaagcgaggactgacggatacgacggtctcgcgcaaggtcgacggcatgcctcatcgtgtgctcaacaccgacctggtcaacagcctcgaaggttccagctatgcaaccggtttgatcgctgctgccaagaacgccaccaagttcaaggcaatgacaggtatgaagtggtcgacgctcgccaagga

SEQ ID NO: 2 - DNA, 909 bp, Rhodococcus erythropolis
  CDS (1)..(906): ipdA
atggctagcaagcgtgacaagacgaagtcgctcgacgaggtagtcggcgagctccgcagcggaatgacgatcggcttgggcggctggggatctcgccgcaagccgatggccttcgtgcgtgcgattctgcgttcggacatcaaggacctgaccgtggtcacttacggcggaccggatctcgggctgctgtgctcggccggcaaggtcaagaaggcgtactacggtttcgtgtccctggactccgctccgttctacgatccgtggtttgccaaggcacgcaccgccggtgagatcgaagtccgcgagatggacgagggaatggtcaagtgcggcctcgaagccgctgccgctcgcctccctttcctcccgatccgcgcgggcctgggttcggacgttcgcaacttctggggcgacgaactcaagaccgtcacctccccgtacccagaggccgacggccgttccgagaccttgatcgcgatgcctgcactcaacctcgatgcgtcgttcgttcacctcaatctcggtgacaagcacggcaatgccgcgtacaacggtgtcgacccgtacttcgacgacctgtactgcatggccgccgaaaagcgttacgtctccgtcgaacgcattgtcgagaccgaggaactggtcaaatccgttccactgcagaatcttctgctcaaccgcatgatggtcgacgccgtcgtcgaggccccgaacggtgcccacttcacgttggccggcgaaagctacggccgcgacgagaagttccagcgtcactacgccgaggccgccaagacccccgagtcgtggcagacgttcgtcgacaccttcctctccggcagcgaagaggactaccaagccgcagtaaagaagtttgccgattcatcaaaggcaggggagcaggcaaaatga

SEQ ID NO: 3 - protein, 302 aa, Rhodococcus erythropolis
MASKRDKTKSLDEVVGELRSGMTIGLGGWGSRRKPMAFVRAILRSDIKDLTVVTYGGPDLGLLCSAGKVKKAYYGFVSLDSAPFYDPWFAKARTAGEIEVREMDEGMVKCGLEAAAARLPFLPIRAGLGSDVRNFWGDELKTVTSPYPEADGRSETLIAMPALNLDASFVHLNLGDKHGNAAYNGVDPYFDDLYCMAAEKRYVSVERIVETEELVKSVPLQNLLLNRMMVDAVVEAPNGAHFTLAGESYGRDEKFQRHYAEAAKTPESWQTFVDTFLSGSEEDYQAAVKKFADSSKAGEQAK

SEQ ID NO: 4 - DNA, 756 bp, Rhodococcus erythropolis
  CDS (1)..(753): ipdB
atgagcgaatccaccgtcacccgcgcagagtacgttgttctcgcgtgcgctgaaatcttctccggtgcaggcgaaatcatggccagcccgatgtcgacgtcgtccaccatcggcgctcgcctggctcggctcaccaccgaacccgacctgctgatcaccgatggtgaagccctcattctcgaggacaccccggcagtcggaacgaagggccccatcgaaggatggatgcctttccgcaaggtgttcgacgtcgtcgcttcgggccgtcgccacgtagtcatgggcgccaatcagctcgatcgccacggcaaccagaacctctccgccttcggcccgcttcagcagccgacgcgtcagatgttcggtgtgcgcggcgccccgggcaacaccatcaaccacgcgacgagctacttcgtccccaagcactccaagcgagtgttcgtcgacaaggtcgacgtggtgtgcggtgtcggctacgaccagatcgatcccgagaacccggcatacaagtacctgaacatcccccgcgttgtcaccaacctcggtgtcttcgacttcggtggaccgggaaacaccttccgcgcgctgagcctccatcccggcgtcaccgccgaagaggtagccgagaacacctcgttcgaggtagccggactcgccgaggccggtatcacccgtgaccccaccgccgaagagctccacctcattcgcgagaccctcgatccgcgcaaccttcgggaccgtgaggtctcggcatga

SEQ ID NO: 5 - protein, 251 aa, Rhodococcus erythropolis
MSESTVTRAEYVVLACAEIFSGAGEIMASPMSTSSTIGARLARLTTEPDLLITDGEALILEDTPAVGTKGPIEGWMPFRKVFDVVASGRRHVVMGANQLDRHGNQNLSAFGPLQQPTRQMFGVRGAPGNTINHATSYFVPKHSKRVFVDKVDVVCGVGYDQIDPENPAYKYLNIPRVVTNLGVFDFGGPGNTFRALSLHPGVTAEEVAENTSFEVAGLAEAGITRDPTAEELHLIRETLDPRNLRDREVSA

SEQ ID NO: 6 - DNA, 915 bp, Rhodococcus erythropolis
  CDS (1)..(912): ipdF
atgagtggattggtcgacggacgcgtagtcatcatcaccggggctggtcgcgggatcggacgtgcgcatgccttggcgtttgctgccgagggcgccaaggtcgtcgtcaacgacatcggtgcgggtgccgacggttccgagaccggtgagagtccggccgagcaggttgtcgcggagatcatcgccgccggtggccaagcagtggtcaacggagacgacgtcgccgactgggcgggcgccgagaatctgatcaagaccgcgatcgacaccttcggtggcctggacgtactcgtgaacaacgcgggcttcctgcgggaccgcatgctggtcggcatgagcgaaggcgagtgggacgcggtgattcgcgtgcacctcaaggggcacttcgcgccgctgcgtcacgctgctgcctactggcgcgctgaggccaaggcaggcaagaccgtcgacgcacgcatcatcaacaccagttcgggtgcaggactgcaaggttcgatcggccagggtaactacgctgcggcgaaggccggtatcgccgagatgaccatccaggccgcagccgagctcaagaattacggcgtcagtgtcaacgcgatcgcgcctgctgcgcgcacgcgcatgaccgtcggcgcgggtggggcgatggccgagtcgatggcagctcccgaagagggcttcgatgccatggcgcccgagaacatctcgccgctggtcgtctggctcggtagcgccgagtccaaggacgtcacggggcgggtgttcgaggtcgagggcggaaagatcaccgtcgcggagggctggcgtcacggcccgagcgaggacaagggtgaccgttgggacccgaaggagatcgggcccgtcgtggcaacgctgttggagaaggcggagattccgacgccggtgtacggagcgtag

SEQ ID NO: 7 - protein, 304 aa, Rhodococcus erythropolis
MSGLVDGRVVIITGAGRGIGRAHALAFAAEGAKVVVNDIGAGADGSETGESPAEQVVAEIIAAGGQAVVNGDDVADWAGAENLIKTAIDTFGGLDVLVNNAGFLRDRMLVGMSEGEWDAVIRVHLKGHFAPLRHAAAYWRAEAKAGKTVDARIINTSSGAGLQGSIGQGNYAAAKAGIAEMTIQAAAELKNYGVSVNAIAPAARTRMTVGAGGAMAESMAAPEEGFDAMAPENISPLVVWLGSAESKDVTGRVFEVEGGKITVAEGWRHGPSEDKGDRWDPKEIGPVVATLLEKAEIPTPVYGA

SEQ ID NO: 8 - DNA, 28 bp, Artificial
  P1 (XbaI) Forward primer
gcgtctagactgcgagccgagggacgcg

SEQ ID NO: 9 - DNA, 30 bp, Artificial
  P2 (BamHI) reverse primer
gcgggatccgtccgaacgcagaatcgcacg

SEQ ID NO: 10 - DNA, 29 bp, Artificial
  P3 (BamHI) forward primer
gcgggatccctcgccgaggccggtatcac

SEQ ID NO: 11 - DNA, 29 bp, Artificial
  P4 (SmaI) reverse primer
gcgggatccctcgccgaggccggtatcac

SEQ ID NO: 12 - DNA, 28 bp, Artificial
  IpdF-F forward primer
atacatatgagtggattggtcgacggac

SEQ ID NO: 13 - DNA, 31 bp, Artificial
  IpdF-R reverse primer
ataggatccctacgctccgtacaccggcgtc

(19) United States
(12) Patent Application Publication
(10) Pub. No.: US 2009/0186390 A1
(43) Pub. Date: Jul. 23, 2009

(54) Method for the Production of Modified Steroid Degrading Microorganisms and their Use

(75) Inventors: Robert van der Geize, Veendam (NL); Gerda Hessels, Groningen (NL); Lubbert Dijkhuizen, Zuidlaren (NL)

Correspondence Address: ORGANON USA, INC.;c/o Schering-Plough Corporation, 2000 Galloping Hill Road, Mail Stop: K-6-1, 1990, Kenilworth, NJ, 07033, US

(73) Assignee: N.V. Organon

(21) Appl. No.: 12/194,903

(22) Filed: Aug. 20, 2008

Related U.S. Application Data
(60) Provisional application No. 60/957,030, filed on Aug. 21, 2007.

(51) Int. Cl. C12P 15/00; C12N 15/01; C12N 1/20; C07K 14/00; C07H 21/02

(52) U.S. Cl. 435/127; 435/448; 435/252.1; 530/350; 536/23.1

(57) ABSTRACT

A method is described to construct genetically modified strains of steroid degrading micro-organisms wherein the method comprises inactivation of at least one gene involved in methylhexahydroindanedione propionate degradation. Strains with (multiple) inactivated steroid degrading enzyme genes according to the invention can be used in the accumulation of steroid intermediates. Accumulation products are for example 3aα-H-4α(3′-propionic acid)-7aβ-methylhexahydro-1,5-indanedione (HIP), 3-hydroxy-9,10-secoandrosta-1,3,5(10)-triene-9,17-dione (3-HSA), 1,4-androstadiene-3,17-dione (ADD) and 3aα-H-4α(3′-propionic acid)-5α-hydroxy-7aβ-methylhexahydro-1-indanone-δ-lactone (HIL).

This application is a non-provisional application that claims priority under 35 U.S.C. § 119(e) of provisional application U.S. Ser. No. 60/957,030 filed Aug. 21, 2007, the contents of which are hereby incorporated by reference in its entirety.

The invention relates to a method to prepare genetically modified micro-organisms having inhibited capacity for nucleus degradation of steroids, to the use of such micro-organism in steroid accumulation as well as to said modified micro-organisms.

The ability to degrade steroids is widespread in actinobacteria and requires a set of enzymes degrading the side-chain and the steroid nucleus structure. Rhodococcus species are well-known in the art for their large catabolic potential. Several Rhodococcus species are able to degrade natural phytosterols, which are inexpensive starting materials for the production of bioactive steroids. For instance, it is known that Rhodococcus strains treated with mutagens and/or incubated with enzyme inhibitors convert sterols into 4-androstene-3,17-dione and 1,4-androstadiene-3,17-dione.

It is further known, that methylhexahydroindanedione propionate (HIP; 3aα-H-4α(3′-propionic acid)-7aβ-methylhexahydro-1,5-indanedione) and 5-hydroxy-methylhexahydroindanone propionate (HIL; 3aα-H-4α(3′-propionic acid)-5α-hydroxy-7aβ-methylhexahydro-1-indanone-δ-lactone) are formed during the microbial degradation of steroids and sterols by actinobacteria (FIG. 1), e.g. by Rhodococcus equi, Nocardia restricta, Nocardia corallina, Streptomyces rubescens and Mycobacterium fortuitum. Reportedly, HIL formation has also been observed during deoxycholic acid degradation by Pseudomonas sp.
HIP and HIL are valuable staring compounds for the synthesis of medically important steroids, such as 19-norsteroids.

Previous studies have shown that in the steroid catabolic pathway, degradation of intermediate HIP presumably occurs via a β-oxidation mechanism. The first step in H P degradation in Rhodococcus equi is assumed to be an ATP-dependent CoA activation of HIP, followed by a reduction of the 5′-keto moiety of HIP-CoA by a HIP-reductase, resulting in the formation of HIL-CoA (FIG. 1). Further it is known from literature that in HIP degradation CoA activation is a prerequisite prior to reduction. Microbial CoA-transferases are usually comprised of two pairs of α and β subunits, forming an α₂β₂ enzyme complex, encoded by two separate genes. The crystal structure of glutaconate CoA transferase of Acidaminococcus fermentans has been solved and reported in literature, and further a glutamate residue in the β subunit of glutaconate CoA transferase of A. fermentans and propionate CoA transferase of Clostridium propionicum has been identified as catalytic residue.

Recently, two gene clusters involved in testosterone degradation have been identified in Comamonas testosteroni TA441, one of which contains ORFs suggested to be involved in HIP degradation (Horinouchi, M. et al. Microbiology 147: 3367-3375 (2001), and Biochem Biophys Res Comm 324: 597-604 (2004)). The specific genes for HIP degradation, however, are not known.

The present invention relates to the identification of three genes in Rhodococcus erythropolis SQ1 involved in methylhexahydroindanedione propionate degradation (ipd); two of these genes encode a HIP CoA transferase (ipdA and ipdB), and one gene encodes a putative HIL-(3′α-hydroxypropionyl)-CoA dehydrogenase (ipdF). According to one aspect of the present invention the nucleotide sequences of the ipdA gene, ipdB gene and ipdF gene of R. erythropolis SQ1 have been provided as a gene cluster (SEQ ID NO:1). The present invention also includes DNA sequences comprising nucleotides 1814-2722 of SEQ ID NO:1 (ipdA), nucleotides 2719-3474 of SEQ ID NO:1 (ipdB), and nucleotides 927-13 of SEQ ID NO:1 (ipdF). Furthermore, the present invention includes an IpdA protein comprising the amino acid sequence SEQ ID NO:3 or orthologues therefrom, an IpdB protein comprising the amino acid sequence SEQ ID NO:5 or orthologues therefrom, and an IpdF protein comprising the amino acid sequence SEQ ID NO:7 or orthologues therefrom. Preferably these orhologues belong to the genus Rhodococcus but also related genera belonging to the family of Actinomycetes, such as Nocardia, Corynebacterium, Mycobacterium, and Arthrobacter, can be used. More particularly, the ipdA protein is encoded by nucleotides 1814-2722 of SEQ ID NO:1. The ipdB protein is encoded by nucleotides 2719-3474 of SEQ ID NO:1. The ipdF protein is encoded by nucleotides 927-13 of SEQ ID NO:1.

Finally, the invention includes DNA sequences encoding the above-mentioned IpdA protein, IpdB protein, and an IpdF protein.

Primarily, the present invention relates to a method to construct a genetically modified strain of a steroid-degrading micro-organism, wherein the method comprises inactivation of at least one gene involved in methylhexahydroindanedione propionate degradation. In particular, the method comprises inactivation of multiple genes D involved in methylhexahydroindanedione propionate degradation. Another embodiment of the invention relates to a method wherein at least one gene encoding a HIP CoA transferase is inactivated, and particularly wherein the HIP CoA transferase genes ipdA, encoding the α-subunit of HIP CoA transferase, and ipdB, encoding the β-subunit of HIP CoA transferase, are inactivated.

A further embodiment relates to a method to construct a genetically modified strain of a steroid-degrading micro-organism wherein a gene encoding a HIL-(3′α-hydroxypropionyl)-CoA dehydrogenase (ipdF) is inactivated.

Still another embodiment is a genetically modified micro-organism wherein at least one gene involved in methylhexahydroindanedione propionate degradation has been inactivated according to the present invention. Preferred are micro-organisms belonging to the family of Actinomycetes. More preferred are micro-organisms belonging to the genus Rhodococcus. Most preferred embodiments are the strains Rhodococcus erythropolis RG37 and Rhodococcus erythropolis RG33.

The micro-organism strains Rhodococcus erythropolis RG37 and Rhodococcus erythropolis RG33 have been deposited at the Deutsche Sammlung von Mikroorganismen und Zellkulturen GmbH (DSMZ), Mascheroder Weg 1b, D-38124 Braunschweig, Germany under the accession numbers DSM 18157 and DSM 18156 respectively. These deposits have been made under the terms of the Budapest Treaty.

According to another aspect of the present invention micro-organisms possessing gene inactivation according to the present invention can be used in the preparation of intermediates of the steroid catabolic pathway by accumulation thereof. When 9α-hydroxy-4-androstene-3,17-dione (9OHAD) is incubated with a mutant strain in which HIP CoA transferase is inhibited (e.g. by inactivation of the ipdAB genes) accumulation of HIP occurs, a starting material for the synthesis of 19-norsteroids. Also, in this conversion 3-hydroxy-9,10-secoandrosta-1,3,5(10)-triene-9,17-dione (3-HSA) is formed as accumulation product. Therefore, another embodiment of the present invention is the use of a genetically modified strain of a micro-organism wherein the ipdAB genes are inactivated according to the present invention, in the preparation of 3aα-H-4α(3′-propionic acid)-7aβ-methylhexahydro-1,5-indanedione (HIP) and/or 3-hydroxy-9,10-secoandrosta-1,3,5(10)-triene-9,17-dione (3-HSA) by growing said strain on a culture medium comprising 9OHAD.

Another embodiment of the invention relates to the use of a genetically modified strain of a micro-organism wherein the ipdAB genes are inactivated according to the invention, in the preparation of 1,4-androstadiene-3,17-dione (ADD) by growing said strain on a culture medium comprising 4-androstene-3,17-dione (AD).

A further embodiment of the present invention is the use of a genetically modified strain of a micro-organism wherein the ipdF gene is inactivated according to the present invention, in the preparation of HIL by growing said strain on a culture medium comprising AD. Another embodiment is the use of a genetically modified strain of a micro-organism wherein the ipdF gene is inactivated according to the present invention, in the preparation of 3aα-H-4α(3′-propionic acid)-7aβ-methylhexahydro-1,5-indanedione (HIP) and/or 3-hydroxy-9,10-secoandrosta-1,3,5(10)-triene-9,17-dione (3-HSA) by growing said strain on a culture medium comprising 9OHAD.

Inactivation of genes is a powerful tool for analysis of gene function and for introduction of metabolic blocks. Gene disruption with a non-replicative vector carrying a selective marker is the commonly used method for gene inactivation. Construction of strains with desirable properties via metabolic pathway engineering approaches, however, may require the stepwise inactivation or replacement of several genes. This is only possible when a suitable strategy for introduction of unmarked gene deletions or gene replacements, allowing infinite rounds of metabolic engineering without being dependent on multiple markers, is available. Methods for introduction of unmarked gene deletions in actinobacteria, in particular in the genus Rhodococcus have been reported e.g. in WO 01/31050.

An advantage of unmarked mutation is that it allows the repetitive introduction of mutations in the same strain. Foreign DNA (vector DNA) is removed in the process of introducing the mutation. Newly introduced vector DNA, for the introduction of a second mutation, therefore cannot integrate at the site of the previous mutation (by homologous recombination between vector DNA's). Integration will definitely happen if vector DNA is still present in the chromosome and will give rise to a large number of false-positive integrants. The system enables the use of a sole antibiotic gene for the introduction of an infinite number of mutations. Unmarked mutation also allows easy use in the industry because of the absence of heterogeneous DNA allowing easy disposal of fermentation broth.

Gene inactivation by gene deletion enables the construction of stable, non-reverting mutants. Especially small genes (<500 bp) are inactivated more easily by gene deletion compared to gene disruption by a single recombination integration. Gene deletion mutagenesis can also be applied to inactivate a cluster of several genes from the genome. The gene deletion mutagenesis strategy can be applied also for gene-replacement (e.g. changing wild type into mutant gene).

The preferred strain for mutagenesis of the catabolic steroid ipd genes is Rhodococcus erythropolis. However, unmarked gene deletion of similar genes in other species, genetically accessible by e.g. conjugation or electrotransformation, is conceivable if the molecular organization is the same (or similar) as in R. erythropolis SQ1. Preferably these species belong to the genus Rhodococcus but also related genera belonging to the family of Actinomycetes, such as Nocardia, Mycobacterium, and Arthrobacter, can be used.

As a further embodiment of the present invention, for further gene inactivation, the same methods may be used again, or, alternatively, UV irradiation or chemical means such as nitroguanidine or diepoxyethaan may be used. Methods to introduce gene mutations in that way are well known in the art.

Also, methods to construct vehicles to be used in the mutagenesis protocol are well known (Sambrook et al., Molecular Cloning: a Laboratory Manual, Cold Spring Harbor Laboratory Press, Cold Spring Harbor, latest edition). Furthermore, techniques for site directed mutagenesis, ligation of additional sequences, PCR, sequencing of DNA and construction of suitable expression systems are all, by now, well known in the art. Portions or all of the DNA encoding the desired protein can be constructed synthetically using standard solid phase techniques, preferably to include restriction sites for ease of ligation.
Modifications and variations of the method for introducing disrupted gene mutations or unmarked gene deletion as well as transformation and conjugation will be obvious to those skilled in the art from the foregoing detailed description of the invention. Such modifications and variations are intended to come within the scope of present application.

### LEGENDS TO THE FIGURES

FIG. 1. Scheme showing the proposed sterol/steroid (AD and cholesterol) catabolic pathways of R. erythropolis SQ1 and degredation of the HIP propionate side chain by β-oxidation. The ipd genes putatively involved in HIP degradation are indicated between brackets. The ipdAB genes, encoding the HIP-CoA transferase, and ipdF, encoding the HIL-(3′α-hydroxypropionyl)-CoA dehydrogenase, were deleted in parent strain SQ1, resulting in strain RG 37 and strain RG 33 respectively. The kshAB genes encode the two-component enzyme system 3-ketosteroid 9α-hydroxylase (KSH). The kshB gene is involved in both cholesterol and AD degradation (van der Geize R. et al.: Mol. Microbiol. 45:1007-1018 (2002)). The kstD and kstD2 genes encode 3-ketosteroid Δ1-dehydrogenases (van der Geize et al.: Microbiology 148: 3285-3292 (2002)).

FIG. 2. Schematic overview of an 11 kb genomic DNA fragment of R. erythropolis strain SQ1, containing the ipd gene cluster. Also shown are several pRESQ derived constructs (Table 1) used in functional complementation experiments of HIL growth deficient UV-mutant strains AP10 and AP20. PCR primers used to construct gene deletion mutant RG37 are indicated as P1-P4. IpdF-F and IpdF-R are PCR primers used to check ipdF gene deletion in RG33.

FIG. 3. Degradation of HIL (0.5 mg•mL⁻¹) in glucose (20 mM) mineral medium by parent strain SQ1 (closed circles), ipdAB mutant strain RG37 (triangles) and ipdF mutant strain RG33 (open circles).

FIG. 4. Gas chromatograms of samples taken 72 h after addition of AD, 9OHAD or HIP from cultures of (A) ipdAB mutant strain RG37, (B) ipdF mutant strain RG33 and (C) parent strain SQ1, following growth to late exponential phase in glucose (20 mM) mineral medium. Numbers above peeks indicate the following compounds: 1, HIP; 2 HIP; 3, AD; 4, 3-HSA; 5, ADD. Identities of compounds were verified using authentic samples.

A person skilled in the art will understand how to use the methods and materials described and referred to in this document in order to construct micro-organisms according to the present invention.

The following examples are illustrative for the invention and should in no way be interpreted as limiting the scope of the invention.

### EXAMPLES

(A) Isolation of UV-induced Mutants of R. erythropolis SQ1 Blocked in HIL Degradation
HIL growth deficient mutants (HIL⁻) of R. erythropolis SQ01 growing well on mineral glucose agar plates, were selected following UV mutagenesis. Mutants with a glucose⁺/HIL⁻ growth phenotype were selected. Bioconversion experiments were subsequently performed to identify mutants that were blocked in the first step of HIL degradation. It was found that mutant AP10 degraded HIL very slowly, while mutant AP20 was completely blocked in HIL degradation. Strains AP10 and AP20 were selected for functional complementation with a genomic library of R. erythropolis to identify the genes encoding the first steps in HIL degradation.

### (B) Molecular Characterization of the ipd Gene Cluster Following Functional Complementation of HIL Growth Negative Mutants AP10 and AP20

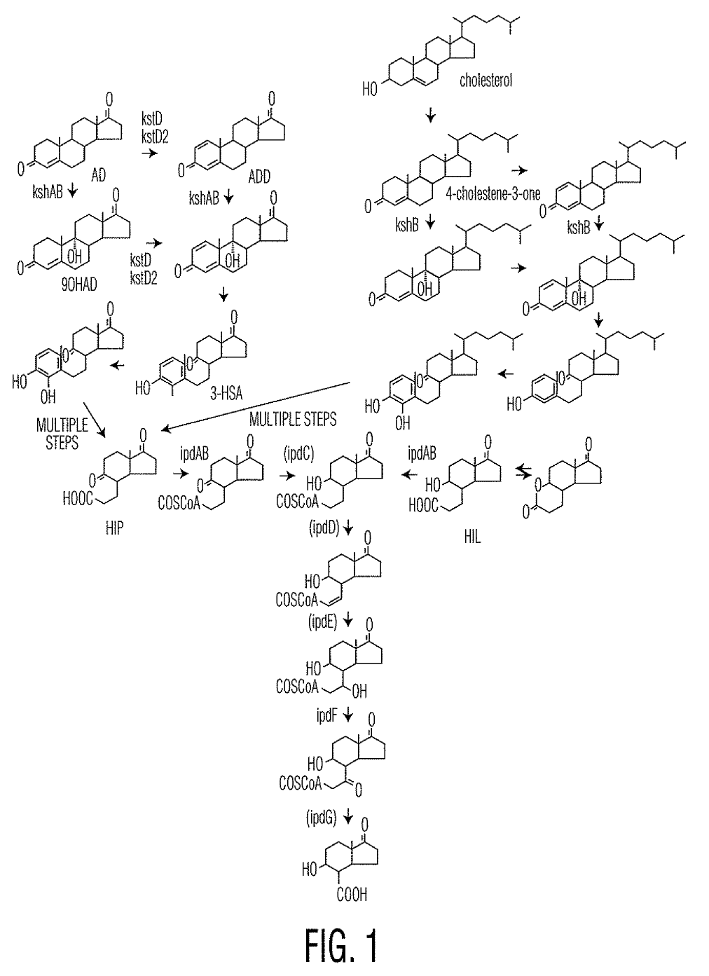

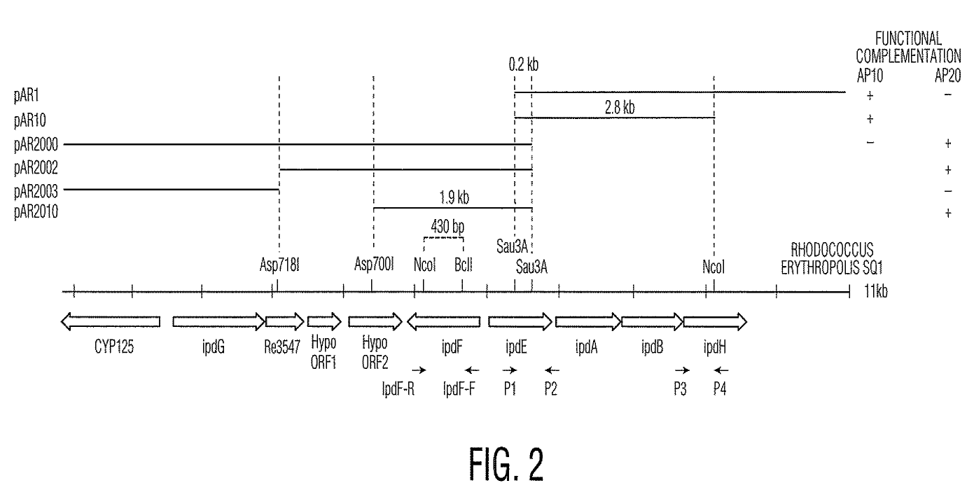

A genomic library of R. erythropolis, constructed in the Rhodococcus-E. coli shuttle vector pRESQ (van der Geize R. et al.: Mol Microbiol. 45:1007-1018 (2002)), was introduced into mutant strains AP10 and AP20 to complement its mutant HIL⁻ growth phenotype. This resulted in the isolation of two plasmids, pAR1 and pAR2000, that were able to restore growth of AP10 and AP20, respectively. Attempts for cross-complementation, introducing pAR1 into mutant AP20 and pAR2000 into mutant AP10, did not restore growth on HIL mineral agar plates, indicating that different genes had been inactivated in these two mutants (FIG. 2). Restriction analysis of pAR1 and pAR2000 confirmed the uniqueness of both plasmids, revealing different restriction patterns. Subsequent nucleotide sequence analysis revealed overlap of approximately 0.2 kb between both plasmids, resulting in a total contiguous sequence of about 11 kb (GC content, 62.1%). The contiguous DNA sequence revealed a total number of 10 ORFs. Database similarity searches indicated that several genes were homologous to genes involved in β-oxidation.
The genes were tentatively designated ipdA to ipdH, because of their expected involvement in methylhexahydroindanedione propionate degradation (Table 2, FIGS. 1 and 2). The ipdABH genes appear to be translationally coupled (ATGA start-stop codons), probably comprising an operon. This operon most likely includes the ipdE gene as well, since the start codon of ipdA is separated by only 7 nt from the stop codon of ipdE. The putative ipdEABH operon structure is highly conserved among many actinomycetes and, to a lesser extent, in C. testosteroni TA441.
(C) Molecular Characterization and Unmarked In-frame Gene Deletion Indicate that ipdAB Encode a CoA-transferase Involved in HIP and HIL Degradation
A series of sub-clones of pAR1 were constructed in pRESQ in order to determine which genes had been inactivated by the UV treatment in mutant AP10 (FIG. 2). A 2.8 kb DNA fragment of the insert of pAR1 (FIG. 2), carrying ipdA and ipdB as intact genes, was cloned into pRESQ (pAR10, Table 1, FIG. 2) and introduced into AP10. This fragment could functionally complement mutant AP10, indicating that either ipdA or ipdB had become inactivated in AP10. The ipdA and ipdB genes encode proteins of 302 amino acids (ipdA, Mw 33.2 kDa) and 251 amino acids (IpdB, Mw 27.1 kDa), respectively. Database similarity searches revealed that IpdA contains the Pfam01144 signature of Coenzyme A transferases (http://www.sanger.ac.uk/Software/Pfam/) as well as the COG1788 signature (http://www.ncbi.nlm.nih.gov/Structure/cdd/cdd.shtml) of AtoD, the α subunit of acyl CoA:acetate/3-ketoacid CoA transferase of E. coli. IpdB furthermore contains the COG2057 signature of AtoA, the β subunit of acyl CoA:acetate/3-ketoacid CoA transferase of E. coli. IpdA and IpdB also share amino acid sequence similarity with GctA (25% identity, Mw 35.7 kDa) and GctB (25% identity, Mw 29.2 kDa), the α and β subunits of glutaconate CoA-transferase of A. fermentans, respectively (Mack M. et al.: Eur. J. Biochem. 226: 41-51 (1994)). Thus, IpdA and IpdB encode the α and β subunit of a CoA-transferase involved in HIL degradation.

### (D) Construction of Mutant RG37

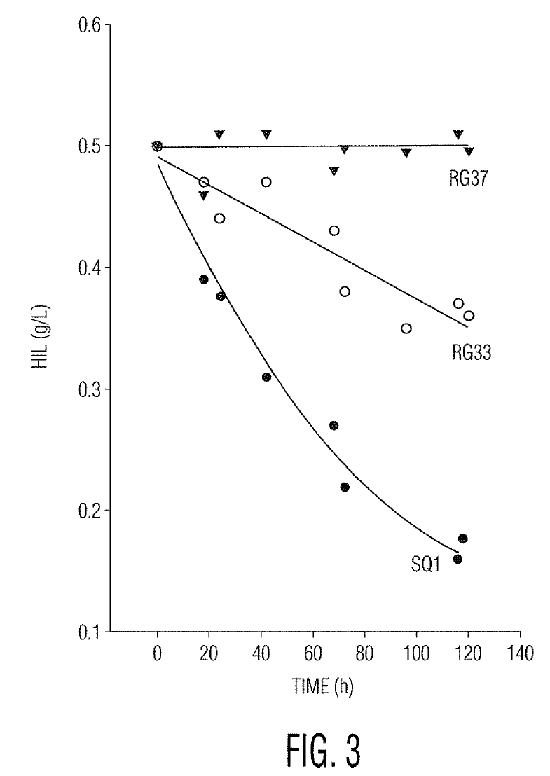

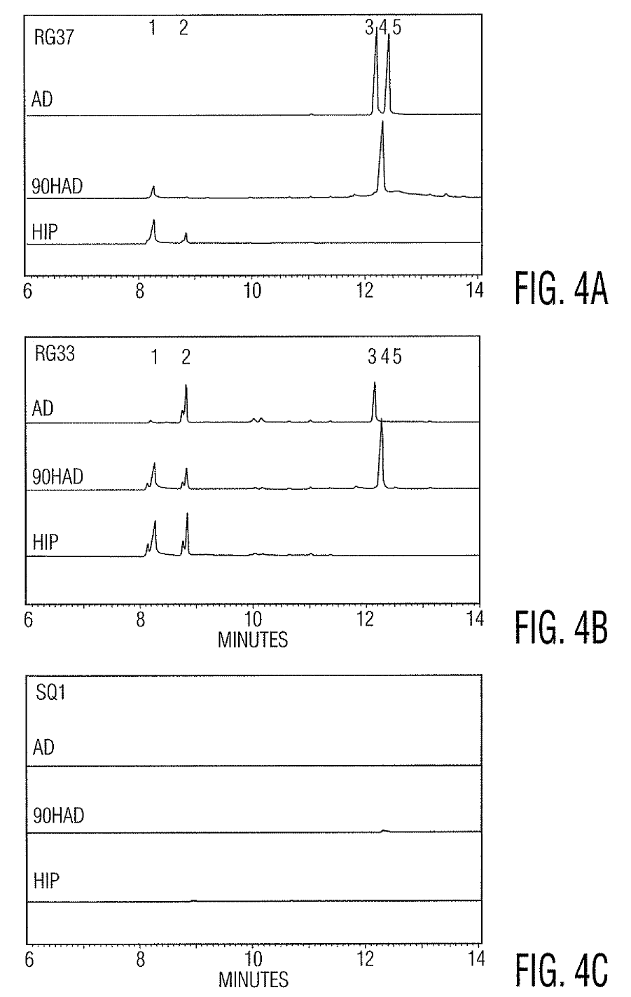

An unmarked ipdAB gene deletion mutant of parent R. erythropolis strain SQ1 was constructed to confirm the involvement of ipdAB in HIL degradation. This mutant, designated R. erythropolis strain RG37, was constructed using mutagenic plasmid pAR31 via the sacB counter-selection method (van der Geize et al.: FEMS Microbiol. Lett. 205:197-202 (2001)). Simultaneous gene deletion of both ipdA and ipdB resulted in a single in-frame ORF remnant of 249 nt in the genome of RG37, encoding the first 46 amino acids of IpdA and the last 36 amino acids of IpdB. Gene deletion was confirmed by PCR using the P1 forward and P4 reverse primers (FIG. 2). Using these primers, PCR products of 1.56 kb and 2.96 kb were found with genomic DNA of mutant strain RG37 and parent strain SQ1, respectively.
(E) Degradation of HIL using Mutant Strain RG37 as Compared to Parent Strain SQ1
Inactivation of the ipdAB genes rendered mutant strain RG37 unable to grow on mineral agar medium supplemented with HIL or HIP as sole carbon and energy source, confirming the involvement of ipdAB in HIL and HIP degradation. Incubation of HIL (0.5 g•L⁻¹) with parent strain SQ1 resulted in a substantial degradation of HIL over a period of five days (FIG. 3). However, no degradation of HIL was observed after 5 days in bioconversion experiments with mutant strain RG37 (FIG. 3). The ipdAB genes thus encode the α and β subunits, respectively, of a HIL CoA transferase, the first step in HIL degradation.
(F) Inactivation of ipdAB Results in Impaired Hydroxylation of Steroid Catabolic Pathway Intermediates
Since HIP and HIL are expected intermediates in steroid degradation (FIG. 1), we studied the ability of mutant strain RG37 to grow on 4-androstene-3,17-dione (AD), 9α-hydroxy-4-androstene-3,17-dione (9OHAD) and cholesterol. Growth of strain RG37 in mineral medium supplemented with either AD, 9OHAD or cholesterol revealed that RG37 was unable to grow on these steroid substrates as sole carbon and energy sources.
We subsequently studied the biotransformation of AD by cultures of RG37 grown to late exponential phase in glucose mineral medium. Strain RG37 was able to partly convert AD into ADD, resulting from 3-ketosteroid Δ1-dehydrogenase (KSTD) activity (van der Geize et al.: Appl. Environ. Microbiol. 66: 2029-2036 (2000) and Microbiology 148: 3285-3292 (2002), FIG. 1). However, AD and ADD were not degraded further and HIP or HIL formation was not observed (FIG. 4A). These results showed that the ipdAB gene deletion had a suppressive effect on AD/ADD 9α-hydroxylation. The mutant phenotype of strain RG37 is similar to the 3-ketosteroid 9α-hydroxylase (KSH) negative mutant phenotypes of the kshA and kshB mutant strains R. erythropolis RG2 and strain RG4, respectively, we previously described (van der Geize R. et al.: Mol. Microbiol. 45:1007-1018 (2002)). The kshA and kshB genes encode the terminal oxygenase component (KshA) and oxygenase-reductase component (KshB) of KSH, respectively, involved in 9α-hydroxylation of AD (forming 9OHAD) and 4-cholestene-3-one. The kshB gene deletion mutant strain RG4 is blocked in 9α-hydroxylation of AD, ADD and 4-cholestene-3-one. Thus, inactivation of ipdAB apparently impairs KSH enzyme activity in R. erythropolis SQ1. Biotransformation of 9α-hydroxylated AD (9OHAD) with mutant strain RG37 resulted in degradation of 9OHAD and the accumulation of intermediates identified as 3-hydroxy-9,10-secoandrosta-1,3,5(10)-triene-9,17-dione (3-HSA) and HIP (FIG. 4A). The accumulation of 3-HSA from 9OHAD was interesting and indicated that also 4-hydroxylation of 3-HSA, the proposed next step in 3-HSA degradation (Horinouchi, M. et al.: Biochem Biophys Res Comm 324: 597-604 (2004), was impaired by the ipdAB deletion. Deletion of the ipdAB genes thus appears to have a marked inhibitory effect on steroid degradation, particularly on the hydroxylation of pathway intermediates, explaining why growth is not observed with strain RG37 using AD and 9OHAD as sole carbon and energy sources. As stated earlier, strain RG37 is also unable to grow on cholesterol as sole carbon and energy source, which is likely due to suppression of 9α-hydroxylation of 4-cholestene-3-one blocking further degradation.
Based on these results we conclude that the ipdAB genes encode the α and β subunit of a CoA transferase with activity towards HIP and HIL. Since HIP is the expected actual substrate of the ipdAB encoded enzyme in the steroid degradation to pathway (FIG. 1), the name HIP CoA-transferase is used.
(G) Molecular Characterization and Unmarked Gene Deletion of ipdF Suggests that IpdF is a HIL-(3′α-hydroxypropionyl)-CoA Dehydrogenase
A set of sub-clones of plasmid pAR2000 in pRESQ was introduced into UV mutant AP20 in order to identify the gene inactivated in this mutant (FIG. 2), A 1.9 kb DNA fragment of the insert of pAR2000 (pAR2010: Table 1, FIG. 2) was still able to functionally complement the AP20 phenotype. The ipdF gene was the sole intact gene on this DNA fragment. We thus concluded that ipdF had been inactivated in the AP20 mutant.

The ipdF gene encodes a protein (IpdF) of 304 amino acids (31.1 kDa). Analysis of the amino acid sequence revealed the presence of a Pfam00106 signature of the short chain dehydrogenase/reductase (SDR) superfamily. Moreover, IpdF contains the glycine motif (Gx(3)GxG (amino acids 14-20) and the Yx(3)K motif (amino acids 171-175) typical for classical SDR proteins (Kallberg et al.: Eur. J. Biochem 269: 4409-4017 (2002)). The highest similarities (71% identity, 82% similarity) were found with hypothetical proteins of the SDR superfamily from several actinomycetes, as well as with ORF27 (53% identity, 68% similarity) of C. testosterone TA 441 (Horinouchi, M. et al.; Microbiology 147; 3367-3375 (2001) and Biochem Biophys Res Comm 324;

597-604 (2004)) In all these bacteria, the genomic location of the corresponding gene was in close proximity to the location of their ipdAB gene orthologues. IpdF furthermore has extensive similarity (37% identity) with the N-terminal (amino acids 1-323) part of mammalian 17β-hydroxysteroid dehydrogenase IV (HSD17B4; Leenders et al.; Eur. J. Biochem, 222; 221-227 (1994)), also known as peroxisomal multifunctonal protein 2 (MFP-2; Dieuaide-Noubhani et al.; Biochem. J. 325; 367-73 (1997)). As the name implies, HSD17B4/MFP-2 is a multifunctional protein (737 amino acids, 80 kDa) exhibiting several enzymatic activities. The N-terminal portion of HSD17B4/MFP-2 is cleaved off as a 32 kDa enzyme, having 17β-hydroxysteroid dehydrogenase and 3-hydroxyacyl-CoA dehydrogenase activities (Adamski et al.: Steroids 62: 159-163 (1997)). Based on these similarities it is assumed that IpdF is the HIL-(3′α-hydroxypropionyl)-CoA dehydrogenase involved in β-oxidation of the propionate side chain of HIL (FIG. 1).

### (H) Construction of Mutant RG33

An ipdF gene deletion mutant strain RG33 was constructed from R. erythropolis SQ1 to confirm the involvement of IpdF in HIP, HIL, and steroid degradation. An internal DNA fragment (0.43 kb) of wild type ipdF gene (915 bp) was deleted (FIG. 2) using pAR2015 (Table 1; see “Experimental procedures” section) as mutagenic plasmid. Following ipdF gene deletion, a frame-shifted ORF remnant of 484 bp, encoding a nonsense protein of 98 amino acids, was introduced, Genuine ipdF gene deletion was confirmed by PCR using ipdF forward (IpdF-F) and reverse (IpdF-R) primers (see “Experimental procedures” section). A PCR product of 499 bp was found with genomic DNA isolated from mutant strain RG33, compared to a 930 bp PCR fragment for wild type ipdF with genomic DNA isolated from parent strain SQ1.
(I) Degradation of HIL using Mutant Strain RG33

Mutant strain RG33 was unable to grow on mineral medium supplemented with HIL (0.5 g•L⁻¹) or HIP (0.5 g•L⁻¹) as sole carbon and energy source. Moreover, degradation of HIL (0.5 g•L⁻¹) was impaired and HIL concentrations decreased more slowly in biotransformation experiments with RG33 over a period of 5 days compared to wild type (FIG. 3).

(J) Inactivation of ipdF Results in HIL Accumulation from AD
Strain RG33 was also not able to grow in mineral liquid medium supplemented with AD, 9OHAD or cholesterol as sole carbon and energy sources, Biotransformation of AD by liquid cultures of strain RG33 grown to late exponential phase in glucose mineral medium revealed that, in contrast to strain RG37, 9α-hydroxylation was not impaired and accumulation of HIL from AD occurs (FIG. 4B). Incubation of RG33 cultures with 9OHAD on the other hand. resulted in the accumulation of 3-HSA, HIP and HIL, indicating that degradation of 9OHAD was affected by ipdF inactivation. The accumulation of the expected substrate of IpdF, HIL-(3′α-hydroxypropionyl) [3OH-HIL], could not be verified with authentic 3OH-HIL. Authentic 3OH-HIL could not be obtained from a commercially source nor synthesized easily. However, the high similarity of ipdF to 3-hydroxyacyl-CoA dehydrogenase domain of mammalian HSD17B4/MFP-2 multifunctional protein in addition to the fact that ipdF is essential for growth on HIP/HIL strongly implies that ipdF encodes HIL-(3′α-hydroxypropionyl)-CoA dehydrogenase.

### Experimental Procedures

### (K) Bacterial Strains, Plasmids and Growth Conditions

Plasmids and bacterial strains used are listed in Table 1. Rhodococcus strains were cultivated at 30° C. and 200 rpm. Complex medium (LBP) contained 1% (wt/vol) bacto-peptone (Difco, Detroit, Mich.), 0.5% (wt/vol) yeast extract (BBL Becton Dickinson and Company, Cockeysville, Md.) and 1% (wt/vol) NaCl. Mineral medium (MM) consisted of 4.65 g L⁻¹ K₂HPO₄, 1.5 g L⁻¹ NaH₂PO₄ H₂O, 3 g L⁻¹ NH₄Cl, 1 g L⁻¹ MgSO₄.7H₂O, and Vishniac trace elements (pH 7). Filter sterilized glucose (20 mM) was added to autoclaved medium. Steroids, HIP, and HIL, supplied by Diosynth bv. (Oss, The Netherlands), were solubilized in DMSO (50 mg.mL⁻¹) and added to autoclaved medium to final concentration of 0.5 g.L⁻¹ for growth experiments and 1 g.L⁻¹ for biotransformation experiments. Cholesterol (1 g.L⁻¹, Sigma) was added as solid to mineral liquid medium, finely dispersed by sonication and subsequently autoclaved. Growth on mineral liquid media was followed spectrophotometrically (AD, 9OHAD, HIP, HIL) or by determination of total protein content of the culture (cholesterol, BioRad protein assay). Sucrose (Suc) sensitivity of Rhodococcus strains was tested on LBP agar supplemented with 10% (w/v) sucrose (LBPS). E. coli strains (Table 1) were grown in Luria-Bertani (LB) broth at 37° C. BBL agar (1.5% (wt/vol)) was added for growth on solid medium.

### (L) General Cloning Techniques

DNA modifying enzymes were purchased from Boehringer (Mannheim, Germany). New England Biolabs (Beverly, Mass.) or Amersham Pharmacia Biotech AB (Uppsala, Sweden) and were used as described by the manufacturer. Isolation of DNA restriction fragments from agarose gels was done using the GeneClean II (Q-BIOgene, Carlsbad, Calif., USA) gel extraction kit according to protocol. All DNA manipulations were done according to standard protocols. PCR was performed under standard conditions using Expand polymerase (Boehringer) unless stated otherwise: 30 cycles of 1 min 95° C. 45 sec 60° C., 1,5 min 72° C. Genomic DNA isolation and colony PCR was performed as described (van der Geize et al.: Appl. Environ. Microbiol. 66: 2029-2036 (2000)) Transformation of Rhodococcus strains for unmarked gene deletion experiments was performed by mobilization of the mutagenic vector from E. coli S17-1 (Table 1) to the Rhodococcus strain by conjugation as described (van der Geize et al.: FEMS Microbiol. Lett. 205:197-202 (2001)).
(M) UV Mutagenesis of R. erythropolis Strain SQ1
UV-induced mutagenesis essentially was done as previously described (van der Geize et al.: FEMS Microbiol. Lett. 205:197-202 (2001)). HIL growth negative mutants, growing well on glucose mineral agar plates, but blocked in growth on mineral agar plates supplemented with 0.5 g.L⁻¹ HIL, were selected for further work.
(N) Functional Complementation of Rhodococcus mutants AP10 and AP20
Electro-competent cells of mutant strains AP10 and AP20 were transformed with R. erythropolis genomic library (van der Geize et al., Mol Microbiol 45:1007-1018, (2002)). Transformations were replica plated onto HIL mineral agar plates (without antibiotic) for screening. Functional complementation of HIL growth negative mutants was observed after approximately 5 days. Plasmid DNA was isolated from the respective Rhodococcus tranformants and used for re-tranformation of the Rhodococcus mutants to check for genuine funstional complemetation by the isolated plasmid.

### Bioconversion Experiments and Analysis by GC, HPLC and TLC

R. erythropolis parent strain SQ1 and mutants were grown in 50 mL glucose (20 mM) mineral medium for 2-3 days (OD₆₀₀>2). Steroids, HIP or HIL were added (1 g.L⁻¹ final concentration) and bioconversion was followed during 5 days (in duplicate). Samples for GC and TLC analysis (0.5 mL) were acidified with 10 μl 10% H₂SO₄. Sample extraction was done using ethylacetate (2 mL). GC analysis was performed on a GC8000 TOP (Thermoquest Italia, Milan, Italy) with AT-5 MS column measuring 30 m by 0.25 mm (inner diameter) and a 0.25 μm film (Alltech, Ill., USA.) and FID detection at 300° C. Chromatographs obtained were analysed using Chromquest V 2.53 software (Thermoquest). For high-performance liquid chromatography (HPLC) analysis, samples were diluted five times with methanol-water (70:30) and filtered (0.45 μm). HPLC analysis was performed on a reversed-phase Lichrosorb 10RP18 (5u) column, measuring 250 by 4.6 mm (Varian Chrompack International, Middelburg, the Netherlands) with UV detection at 254 nm, and a liquid phase of methanol-water (60:40) at 30° C. TLC was done with Kieselgel 60 F₂₅₄ 10×20 cm (Merck, Darmstadt, Germany) developed in toluene/ethylacetate 1:1.

(P) Construction of Mutagenic Plasmids pAR31 and pAR2015 for ipdAB and ipdF Unmarked Gene Deletion
For unmarked in-frame deletion of ipdA and ipdB, plasmid pAR31 (Table 1) was constructed. A 790 bp PCR fragment (PCR product 1), containing part of ipdE and the beginning of ipdA, was obtained using R. erythropolis SQ1 genomic DNA with P1 (Xbal) forward primer
(5′ GCGTCTAGACTGCGAGCCGAGGGACGCG 3′(SEQ ID NO:8)) and P2 (BamHI) reverse primer (5′ GCGGGATCCGTCCGAACGCAGAATCGCACG 3′ (SEQ ID NO: 9)) (FIG. 2). A second PCR fragment (800 bp, PCR product 2), containing the end of ipdB and part of ipdD, was amplified from R. erythropolis SQ1 genomic DNA with P3 (BamHI) forward primer (5′ GCGGGATCCCTCGCCGAGGCCGGTATCAC 3′ (SEQ ID NO: 10)) and P4 (Smal) reverse primer (5′ GCGCCCGGGCTTGCGCGAGACCGTCGTATC 3′ (SEQ ID NO: 11)). Underlined restriction sites, also indicated between brackets for each primer, were included in the four primers to ensure in-frame linkage of the ipdA start codon and the ipdB stop codon. PCR product 2 was cloned into Smal digested pK18mobsacB (Table 1), resulting in plasmid pAR30. Subsequently, PCR product 1 was digested with Xbal and BamHI and cloned into Xbal/BamHI digested pAR30, resulting in plasmid pAR31. For ipdF gene deletion, a 2.54 kb Xhol fragment of pAR2002 was cloned into pBlueScript II(KS), rendering pAR2013. The internal part (430 bp) of the ipdF gene was deleted by BclI/NcoI digestion of pAR2013 and blunt-ended self-ligation after Klenow treatment. The resulting plasmid (pAR2014) was digested with Xhol and a 2.11 kb DNA fragment. containing the ipdF deletion, was cloned into SalI digested pK18mobsacB, yielding plasmid pAR2015 used for ipdF gene deletion. Genuine ipdF gene deletion was checked by PCR with genomic DNA isolated from strain RG33 with IpdF-F forward primer (5′-ATACATATGAGTGGATTGGTCGACGGAC (SEQ ID NO : 12)) and IpdF-R reverse primer (5′-ATAGGATCCCTACGCTCCGTACACCGGCGTC (SEQ ID NO: 13)).

TABLE 1
Strains and plasmids used in this study.

| Strain or plasmid | Characteristics | Reference/origin |
|---|---|---|
| R. erythropolis SQ1 | Parent strain, HIL⁺ | Quan S. et al., Plasmid 29: 74-79 |
| | | (1993) |
| R. erythropolis RG33 | ipdF mutant of strain SQ1, HIL⁻ | This study |
| R. erythropolis RG37 | IpdAB mutant of strain SQ1, HIL⁻ | This study |
| R. erythropolis AP10 | UV-mutant of strain SQ1, HIL⁻ | This study |
| R. erythropolis AP20 | UV-mutant of strain SQ1, HIL⁻ | This study |
| E. coli DH5α | Host for general cloning steps | Bethesda Res. Lab. |
| E. coli S17-1 | Strain for conjugal mobilization of | Simon et al.: Biotechnology 1: 784-791 |
| | pK18mobsacB derivatives to Rhodococcus | (1983) |
| | strains | |
| pBlueScript(II) KS | bla lacZ | Stratagene |
| pK18mobsacB | aphII sacB oriT (RP4) lacZ | Schäfer et al.: Gene 145: 69-73 (1994) |
| pRESQ | Rhodococcus-E. coli shuttle vector | van der Geize R. et al.: Mol. Microbiol. |
| | | 45: 1007-1018 (2002) |
| pAR1 | pRESQ containing 5.2 kb genomic fragment | This study |
| | of R. erythropolis carrying ipdA and ipdB | |
| pAR10 | pRESQ carrying ipdA and ipdB on a 2.88 kb | This study |
| | NcoI/HindIII fragment of pAR1 (HindIII | |
| | located on cloning vector) | |
| pAR30 | PCR product 2, obtained with primers P3 | This study |
| | and P4 (FIG. 2), cloned in SmaI digested | |
| | pK18mobsacB | |
| pAR31 | PCR product 1, obtained with primers P1 | This study |
| | and P2 (FIG. 2) cloned into XbaI/BamHI | |
| | digested pAR30; used for ipdAB gene | |
| | deletion in SQ1, yielding RG37 | |
| pAR2000 | pRESQ containing 6.1 kb genomic fragment | This study |
| | of R. erythropolis carrying ipdF | |
| pAR2002 | Self-ligation of 9.55 kb fragment of pAR2000 | This study |
| | following Asp718I digestion | |
| pAR2003 | Asp718I fragment (3.2 kb) of pAR2000 | This study |
| | ligated into Asp718I digested pRESQ | |
| pAR2010 | Self-ligation of Asp700I/Asp718I digested | This study |
| | pAR2002 (blunt-ended with Klenow) | |
| PAR2013 | 2.54 kb XhoI fragment of pAR2002 cloned in | This study |
| | XhoI site pBlueScript(II)KS | |
| pAR2014 | Self-ligation of BclI/NcoI digested pAR2013 | This study |
| | (5 kb, blunt-ended with Klenow) | |
| pAR2015 | 2.1 kb XhoI fragment of pAR2014 cloned in | This study |
| | SalI site of pK18mobsac8; used for ipdF | |
| | gene deletion in SQ1, yielding RG33 | |

1. A method to construct a genetically modified strain of a steroid-degrading micro-organism, wherein the method comprises inactivation of at least one gene involved in methylhexahydroindanedione propionate degradation.

2. The method according to claim 1, wherein the method comprises inactivation of multiple genes involved in methylhexahydroindanedione propionate degradation.

3. The method according to claim 1, wherein at least one gene encoding a HIP CoA transferase is inactivated.

4. The method according to claim 3, wherein the HIP CoA transferase genes ipdA and ipdB are inactivated.

5. The method according to claim 1 wherein a gene encoding a HIL-(3′α-hydroxypropionyl)-CoA dehydrogenase (ipdF) is inactivated.

6. The method according to claim 1, wherein any gene is inactivated by UV-irradiation.

7. The method according to claim 1, wherein any gene is deleted by unmarked gene deletion.

8. The method according to claim 1, wherein the micro-organism belongs to the family of Actinomycetesis.

9. The method according to claim 8, wherein the micro-organism belongs to the genus Rhodococcus.

10. The method according to claim 9, wherein the micro-organism is Rhodococcus erythropolis.

11. A genetically modified strain of a micro-organism prepared according to claim 1.

12. The genetically modified strain a-cording to claim 11 being Rhodococcus erythropolis RG37.

13. The genetically modified strain according to claim 11, being Rhodococcus erythropolis RG33.

14. A method for preparing a steroid intermediate, the method comprising
(a) preparing a genetically modified strain of a steroid-degrading micro-organism, which comprises inactivation of at least one gene involved in methylhexahydroindanedione propionate degradation; and
b) adding a steroid starting material to the genetically modified strain of step (a).

15. The method according to claim 14, wherein the steroid intermediate is 3aα-H-4α(3′-propionic acid)-7aβ-methylhexahydro-1,5-indanedione (HIP) and/or 3-hydroxy-9,10-secoandrosta-1,3,5(10)-triene-9,17-dione (3-HSA) and the steroid starting material is 9α-hydroxy-4-androstene-3,17-dione (9OHAD).

16. The method according to claim 14, wherein the steroid intermediate is 1,4-androstadiene-3,17-dione (ADD) and the steroid starting material is 4-androstene-3,17-dione (AD).

17. The method according to claim 14, wherein the steroid intermediate is 3aα-H-4α(3′-propionic acid)-5α-hydroxy-7aβ-methylhexahydro-1-indanone-δ-lactone (HIL) and the steroid starting material is 4-androstene-3,17-dione (AD).

18. An IpdA protein comprising the amino acid sequence SEQ ID NO:3 or orthologues therefrom.

19. An IpdB protein comprising the amino acid sequence SEQ ID NO:5 or orthologues therefrom.

20. An IpdF protein comprising the amino acid sequence SEQ ID NO:7 or orhologues therefrom.

21. The DNA sequence encoding an IpdA protein according to claim 18.

22. The DNA sequence encoding an IpdB protein according to claim 19.

23. The DNA sequence encoding an IpdF protein according to claim 20.

24. A DNA sequence comprising nucleotides 1814-2722 of SEQ ID NO:1.

25. A DNA sequence comprising nucleotides 2719-3474 of SEQ ID NO:1.

26. A DNA sequence comprising nucleotides 927-13 of SEQ ID NO:1.
